(19) United States
(12) Patent Application Publication
(10) Pub. No.: US 2019/0246926 A1
(43) Pub. Date: Aug. 15, 2019

(54) DEVICE FOR DETECTING ATRIAL FIBRILLATION OF A SUBJECT

(71) Applicant: Medtronic, Inc., Minneapolis, MN (US)

(72) Inventors: Nathalie Virag, Cottens (CH); Soren Aasmul, Holte (DK); Catherine Chassard, Sauverny (FR); Richard Cornelussen, Maastricht (NL); Gerard Hill, Valkenburg (NL); Robert Leinders, Urmond (NL); Mirko De Melis, Maastricht (NL); Raphael Schneider, Maastricht (NL); Vasiliki Spyropoulou, Maastricht (NL)

(21) Appl. No.: 16/311,312

(22) PCT Filed: Jun. 22, 2017
(86) PCT No.: PCT/IB2017/053735

§ 371 Date: Dec. 19, 2018

(30) Foreign Application Priority Data
Jun. 23, 2016 (EP) 16175984.0

(51) Int. Cl. A61B 5/046; A61B 5/1455; A61B 5/00

(52) U.S. Cl. CPC A61B 5/046; A61B 5/1455; A61B 5/04012; A61B 5/6826; A61B 5/6831

(57) ABSTRACT

The invention relates to a device for detecting atrial fibrillation of a subject, comprising a member comprising —mechanical connection means to wearably connect the member to a subject; —the member further comprising an optical sensor arranged to measure a signal representing a blood perfusion parameter of the subject; the member comprising the optical sensor and mechanical connection means at such relative positions that when the mechanical connection means connect the member to the subject, the optica! sensor operatively faces the subject. The device is arranged to detect atrial fibrillation of the subject based on the signal measured by the optical sensor.

The invention relates to a device for detecting atrial fibrillation of a subject, comprising a member comprising
mechanical connection means to wearably connect the member to a subject;
the member further comprising an optical sensor arranged to measure a signal representing a blood perfusion parameter of the subject;
the member comprising the optical sensor and mechanical connection means at such relative positions that when the mechanical connection means connect the member to the subject, the optical sensor operatively faces the subject.

Atrial Fibrillation (AF) is an abnormal heart rhythm characterized by a rapid and irregular rhythm in the atria and is asymptomatic in up to one-third of the patients. The potential consequences of untreated AF are an increased risk of heart failure, dementia and stroke. Early detection of atrial fibrillation might allow timely introduction of therapies to protect and mitigate the risks.

Known systems aimed at AF detecting/monitoring are based on ECG recordings. Depending on the patient, different periods of time of AF detection/monitoring are preferred. The detectors for different periods of time come in quite different forms as to how they are worn or carried by the patient.

WO2014/055994A1 discloses an adhesive patch comprising electrodes for ECG-monitoring for a period of two weeks and atrial fibrillation detection.

The patch also comprises an optical pulse oximetry sensor comprising 2 LEDs and a photodiode between the 2 LEDs. The photodiode and the 2 LEDs are covered with a layer of clear silicon to remove any air gap between the 2 LEDs as well as the photodiode and the patient skin to reduce reflection losses and reduce motion artefacts, which introduce noise and are caused by motion of the skin relative to the sensor.

This document also teaches to use an adhesive patch on a chest for conducting pulse oximetry measurements. To reduce noise when conducting pulse oximetry measurements at the chest, it suggests measuring an ECG signal over multiple heart beats, measuring a pulse oximetry signal over multiple heart beats whereby the ECG signal and the pulse oximetry signal are in time concordance over one or more heart beats. The ECG signal is used to determine intervals in the pulse oximetry signals that correspond to full heart beat cycles. A constant and primary periodic component of the pulse oximetry signal over the full heart beat cycles is determined and the oxygen saturation for the constant and primary periodic components is determined.

A drawback of the ECG-based recording system of WO2014/055994A1 for atrial fibrillation detection is that it has to be positioned in one of a set of specific locations on the thorax and that ECG signal quality depends on the correct positioning. Such ECG-based recording systems and positions are less suitable for elderly patients to position the recording systems on themselves in their home environment.

It is an object of the present invention to provide a device which can be applied at other locations than the specific locations for ECG measurements on the thorax of a subject.

The object of the invention is achieved by the invention providing a device for detecting atrial fibrillation of a subject, comprising a member comprising
mechanical connection means to wearably connect the member to a subject;
the member further comprising an optical sensor arranged to measure a signal representing a blood perfusion parameter of the subject;
   the member comprising the optical sensor and mechanical connection means at such relative positions that when the mechanical connection means connect the member to the subject, the optical sensor operatively faces the subject;
   characterized by
the device being arranged to detect atrial fibrillation of the subject based on the signal measured by the optical sensor.

Blood perfusion depends on the heart function. The inventor realized that since blood perfusion depend on the heart function, it can be used to detect atrial fibrillation.

Whereas ECG-based sensors have to be positioned at specific locations on the thorax, optical sensors allow recording at any location that has blood perfusion such as a subject's fingers, toes, ear, nose, forehead, wrist forearm or upper arm. Because the range of locations on a subject's body that has blood perfusion is wider than only the specific locations on the thorax suitable for ECG-recordings, the device gives more freedom for positioning on the subjects body.

The optical sensor can be of a reflective (RPO) or a transmissive (TPO) type.

The subject can be a human or animal (such as a mammal). The subject can either be healthy or a patient.

The mechanical connection means can for instance be
a strap, band or cuff (for instance in case the device takes the form of a wrist worn device),
a ring for wearing on a finger or toe or through skin like an earlobe,
a hook or compressive flange for an ear bud or
an area of adherent material (for instance in case the device takes the form of a patch or a false nail).

In a further embodiment of the invention, the optical sensor is a laser Doppler flowmetry sensor or a pulse oximetry sensor.

Laser Doppler flowmetry sensors and pulse oximetry sensors are well established sensor types suitable for perfusion related measurements.

In a further embodiment of the invention the mechanical connection means comprise an area of adherent material arranged to adhere the member to the subject.

As the mechanical connection means comprise an area of adherent material to adhere the member to the subject, the member can easily and reliably be connected to the subject.

The adherent material can be a medical grade adhesive. Medical grade adhesives are widely known to the person skilled in the art. Preferably a medical grade adhesive is selected for its ability to maintain in intimate contact with the skin without damaging it for several days (up to for instance 5, 7, 10 or 14 days). In addition, the medical grade adhesive can be selected based on its characteristics to be separated from the subject after a period of use.

In a further embodiment of the invention
the adherent material is arranged to adhere to a nail or false nail of the subject;
the optical sensor and the adherent material are arranged at such relative positions that when the adherent material adheres to a nail or false nail of the subject, the optical sensor operatively faces the nail or false nail and is pulled towards the nail or false nail by the adherence.

A nail is less flexible than the skin, is less exposed to mechanical forces than most skin parts as it is on the top of finger tops instead of at the bottom of finger tops and is less subject to extension due to mechanical stress than the skin. The combination of a nail and a false nail is even less flexible and even less subject to extension due to mechanical stress.

As the relative positions of the adherent material and the optical sensor are such that with the adherent material adhering to the (false) nail, the optical sensor operatively faces the (false) nail and is pulled towards the (false) nail by the adherence, this means that the variation of the relative positions is limited.

The combination of adhering the adherent material to the inflexible (false) nail and the limited variation of relative positions means that the relative positions of the optical sensor and the (false) nail are limited.

The more the relative positions of the optical sensor and the (false) nail are limited, the less motion artefacts occur which contributes to obtaining reliable atrial fibrillation determination.

In a further embodiment of the invention
the optical sensor and the adherent material are arranged in such relative positions that in use the relative positions of the nail or false nail and the optical sensor are fixed.

By fixing the relative positions of the (false) nail and the optical sensor, motion artefacts are further minimized thereby further increasing reliability of the atrial fibrillation determination.

In a further embodiment of the invention the device comprises a false nail.

By comprising a false nail, the device can be worn simultaneously with false nails on other fingers or toes, without increasing the thickness of the stack comprising the nail and the device.

In addition, application and removal of a false nail is simple and can be done in a home environment without the need of a physician or technician.

By comprising a false nail, the skin around the nail is not adhered to by adherent material providing comfort to the subject. In addition this prevents skin irritation.

Moreover, as a false nail hardly has an impact on the activities of the subject, the device hardly as an impact on the activities of the subject. Not only is such minimal impact comfortable for the subject, minimal disturbance of the activities also contributes to diagnosing a subject for risks in case the adhesive device would not be applied.

In a further embodiment of the invention, the devices comprises a processing unit arranged to extract RR intervals from the signal and to detect atrial fibrillation based on the extracted RR intervals.

By extracting RR intervals from the signal, information on the heart beats is obtained from the blood perfusion.

In a further embodiment,
the device comprises a plurality of members, the member comprising the optical sensor forming a first member of the plurality of members;
the functionality of the device is split over the plurality of members.

By splitting the functionality over the plurality of members, the device can comprise more components than can be fitted comfortably on a single member.

In a further embodiment of the invention, each of the plurality of members comprises an individual area of adherent material arranged to adhere the corresponding member to the subject.

By comprising an individual area of adherent material, application and removal of the plurality of members is easy. In addition, individual members may be replaced without the need to replace all components.

In a further embodiment of the invention each of the plurality of members comprises a false nail.

Since each of the plurality of members comprises a false nail, all members of the device can easily be applied and removed and worn without the risk of skin irritation. Moreover, as false nails hardly have an impact on the activities of the subject, the device in total hardly as an impact on the activities of the subject. Not only is such minimal impact comfortable for the subject, minimal disturbance of the activities also contributes to diagnosing a subject for risks in case the adhesive device would not be applied.

In a further embodiment of the invention
the plurality of members comprises a second member, the second member comprising a processing unit arranged to process the signal for detecting atrial fibrillation.

By separating the processing and the sensing in different members, the members can remain small which contributes to wear ability and the value of the measurements.

The second member can be formed by a handheld device such as a smartphone. This is advantageous as a smartphone can double to perform more functions for the subject and because the smartphone can forward a detected atrial fibrillation via Internet or mobile data transmission.

Preferred embodiments will now be described by way of example only, with reference to the drawings.

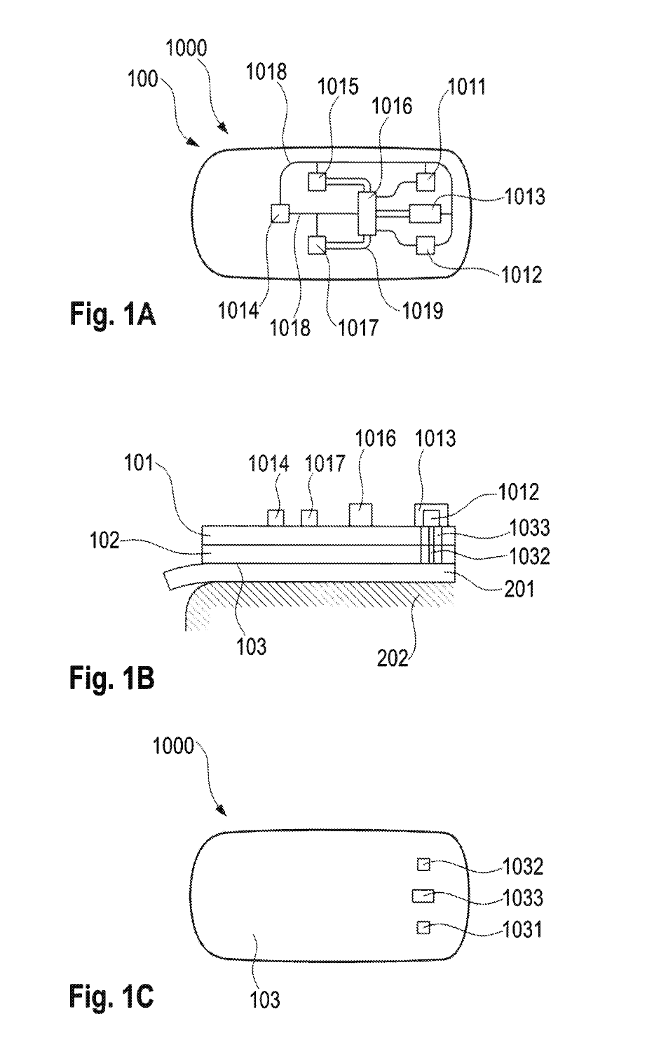

FIG. 1A. is a schematic illustration of a top view of a device according to the invention;

FIG. 1B. is a schematic illustration of a side view of the device according to the invention;

FIG. 1C. is a schematic illustration of a bottom view of the device according to the invention;

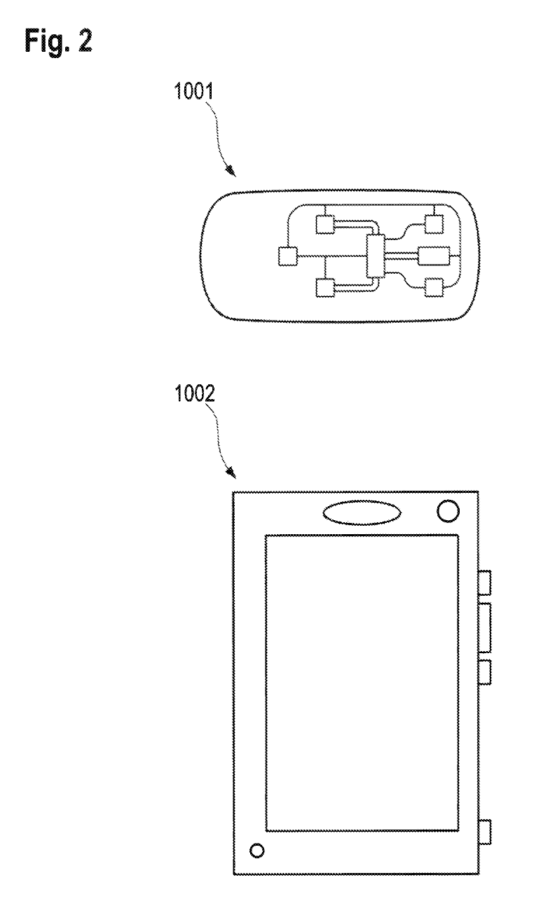

FIG. 2. Is a schematic illustration of the members of a second exemplary embodiment of the invention;

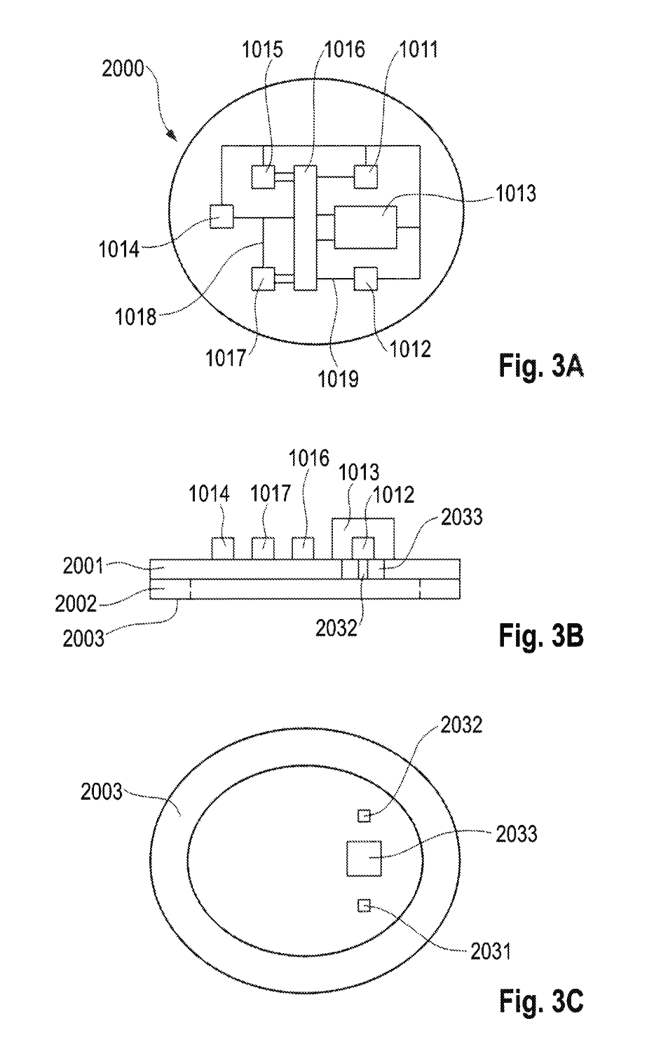

FIGS. 3A, B and C are schematic illustrations of respectively a top view, side view and bottom views of a device according to the invention. They are schematic illustrations of a device according to a fifth exemplary embodiment of the invention; and

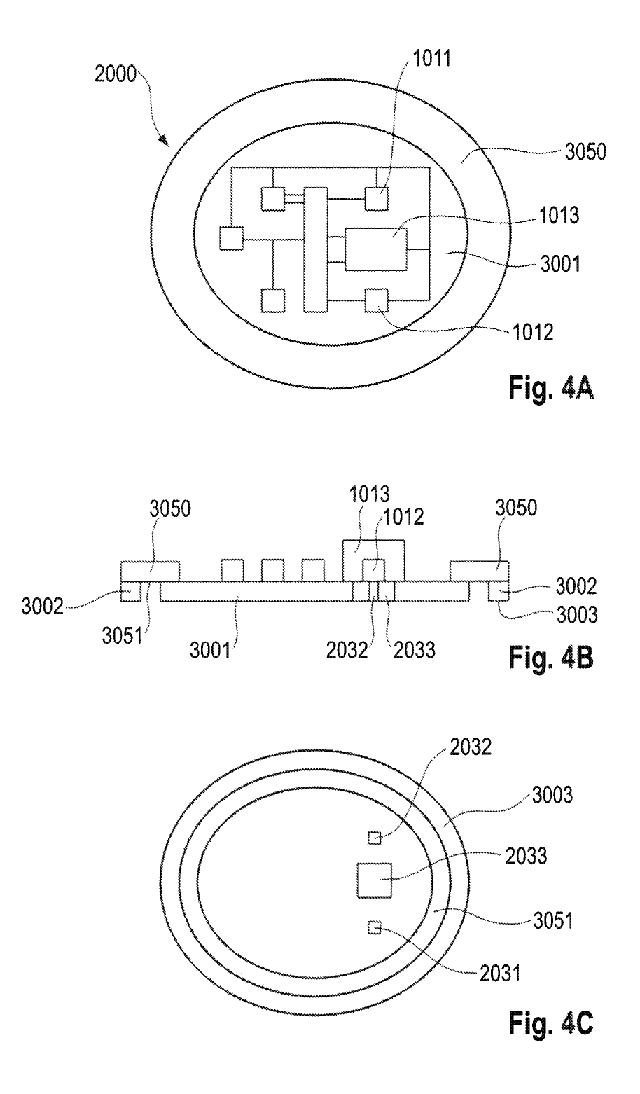

FIGS. 4A, B and C are schematic illustrations of a respectively a top view, side view and bottom views of a device according to the invention. They are schematic illustrations of a device according to a sixth exemplary embodiment of the invention.

In a first exemplary embodiment of the invention a device (100) for detecting atrial fibrillation is aimed for mass screening for asymptomatic atrial fibrillation (AF) patients. More particularly, the device is targeting the population of elderly patients who have undiagnosed AF. As AF is undiagnosed, the persons which will be referred to as subjects. A top view, a side view and a bottom view of the device are shown in FIGS. 1A, 1B and 1C respectively. The bottom view corresponds to viewing the device on the side that is to adhere to the subjects.

The device (100) comprises a member (1000) that comprises a false nail comprising a substrate (101) and an area (103) of adherent material (102), the adherent material (102) forming mechanical connection means. The adherent material (102) is shown (FIG. 1B) to adhere to a nail (201) of a subject. A cross section of the nail (201) is shown in FIG. 1B. In the drawing the nail belt is on the right side. Before it was adhered to the nail (201), the member (1000) was provided with a protective backing to protect the adhesive material (102).

The member (1000) comprises an optical sensor in the form of a pulse oximeter comprising a first LED (1011), a second LED (1012) and a photodiode (1013) all mounted on the substrate (101). The first LED (1011) is arranged to emit red light of 660 nm, the second LED (1012) is arranged to emit infrared radiation of 890 nm. Both the red light and the infrared radiation are arranged to be emitted towards the side that is adhered to the subject. The photodiode is arranged to sense both red light from the first LED (1011) and infrared light from the second LED (1012) reflected from the skin tissue (202) behind the nail (201).

The substrate (101) is covered on the top side with a material that blocks red and infrared radiation. The material is a metal that is applied in a coating.

A first through hole (1031) runs through the substrate (101) and the adherent material (102) between the first LED (1011) and the area (103). The first through hole (1031) is provided to pass the red light from the first LED (1011) to the nail (201).

A second through hole (1032) runs through the substrate (101) and the adherent material (102) between the second LED (1012) and the area (103). The second through hole (1032) is provided to pass the infrared radiation from the second LED (1012) to the nail (201).

A third through hole (1033) runs through the substrate (101) and the adherent material (102) between the photodiode (1013) and the area (103). The third through hole (1033) is provided to pass red light and infrared radiation for the nail (201) and ultimately the skin (202) to the photodiode (1013).

The positions of the first LED (1011), the second LED (1012) and the photodiode (1013) are near an edge of the substrate (101) that is arranged to be aligned with the nail belt. When the adherent material is adhered to the nail (201), the first LED (1012) the second LED (1012) and the photodiode (1013) therefore face the nail (201) and the skin tissue (202) with their respective sides from which red light or infrared radiation is emitted or received. The first LED (1012) the second LED (1012) and the photodiode (1013) and are not placed at a section (2011) of the nail (201) that is not supported by skin tissue (202) but are placed where the nail (201) is supported by skin tissue (202) and therefore operatively face the subject.

Moreover, as the adherent material (102) fixed the substrate (101) of the false nail to the nail (201) of the subject, relative positions of the first LED (1011) and the nail (201) are fixed. Similarly the relative positions of the second LED (1012) and the nail (201) are fixed and the relative positions of the photodiode (1013) and the nail (201) are fixed.

In use the first LED (1011) and the second LED (1012) are fired turn by turn so that the photodiode (1013) can measure the reflected light and radiation at separate time instances to derive the ratio between the red light and infrared radiation in order to obtain a pulsatile signal representing the heartbeat. The photodetector (1013) outputs the obtained signal corresponding to the received amount of red light and infrared radiation.

After firing the first LED (1011) and the second LED (1012) and measuring the reflected light and reflected radiation with the photodiode (1013) an additional measurement is conducted while both the first LED (1011) and the second LED (1012) are not fired, i.e. do not emit red light or infrared radiation. This measurement is conducted to determine background readings of the photodiode (1013). The background readings are subtracted from the readings of the photodiode (1013) taken while either the first LED (1011) or the second LED (1012) is fired to increase the accuracy of the measurements.

As the skilled man will know, pulse oximetry is based on the red and infrared light absorption characteristics of oxygenated and deoxygenated haemoglobin, i.e. on blood perfusion parameters of the subject. The oxygenation state of mixed arterial/venous blood pulsates with heartbeats of the subject.

The member (1000) further comprises a battery (1014), a memory (1015), a controller (1016) and a wireless communication device (1017).

The battery (1014) is connected to the first LED (1011), the second LED (1012), the photodiode (1013), a memory (1015), a controller (1016) and a wireless communication device (1017) to provide them with power via electrically conductive traces (1018) formed on the substrate (101) of the false nail which therefore forms a printed circuit board (PCB). The controller (1016) comprises LED driver electronics and photodiode amplifiers. The battery is arranged to supply the member (1000) with power for one week (7 days). This period is considered optimal for a diagnostic yield in a time period between Holter (typically 24 hours) and implantable loop recorder (which can last up to 3 years), which are ECG based devices for AF detection.

In use, the device boots up after receiving a wake up frequency on an antenna (not shown). The wake up frequency is verified before booting the device to extend the shelve life as long as possible. After booting up, the input for booting is disabled to prevent undesired boots during operation due to noise. While shelved, the device is stored in an aluminium coated bad to prevent undesired booting due to noise.

The controller (1016) is connected to the first LED (1011) and the second LED (1012) via the LED driver electronics, to the photodiode (1013) via the photodiode amplifier, to the memory (1015) and the wireless communication device (1017) via additional electrically conductive traces (1019) to exchange commands and data such as the signal from the photodiode (1013).

The first LED (1011), the second LED (1012), the photodiode (1013), the battery (1014), the memory (1015), the controller (1016) and the wireless communication device (1017), the electrically conducting traces (1018) and the additional electrically conducting traces (1019) together form the optical sensor.

In use the controller (1016) controls the time instances at which the first LED (1011), and the second LED (1012) fire and at which the photodiode (1013) measures. The controller (1016) further arranges that the measurements are stored in the memory (1015) together with corresponding time stamp so that the signal is stored. The wireless communication device (1017) is arranged for a Bluetooth connection with a smart phone.

The controller (1016) is further arranged to subtract background readings from the measurements as explained above. The controller (1016) is also arranged to run an RR extraction algorithm to determine RR intervals. In this embodiment, the RR is determined as the interval between two similar maxima in the ratio between oxygenated and deoxygenated haemoglobin which correspond to the same phase in the heart rhythm of the subject. Such extraction algorithms are well known to the skilled person for ECG time series, for instance from the use in Reveal LINQ™ Insertable Cardiac Monitor (ICM).

The controller (1016) is further arranged to run an AF detection algorithm based on the extracted RR intervals. AF is detected once an RR interval deviates too much from an average of previous RR intervals. AF detection algorithms are well known to the skilled person, such as the one used in Reveal LINQ™. The controller (1016) is arranged to send detected AF occurrences to the smart phone on receiving requests to do so from the smart phone via the wireless communication device (1017).

The device is water resistible allowing showering, washing, etc. In a variant (not shown) of this exemplary embodiment, the member (1000) is covered with a water tight silicone layer on the side that in use faces away from the subject, to protect the electrically conducting traces and the elements mounted on the substrate such as the first LED (1011) and the controller (1016). The silicone layer may also be impenetrable for red light and infrared radiation.

In a second exemplary embodiment, schematically illustrated in FIG. 2, the member (1000) described in the first exemplary embodiment forms a first member (1001) of a plurality of members comprised by the device (100). The first member (1001) differs from the member (1000) described in the first exemplary embodiment in that the controller (1016) is not arranged to run an RR extraction algorithm, is not arranged to run an AF detection algorithm based on the extracted RR intervals and is not arranged to send detected AF occurrences to the smart phone.

Instead, the smart phone forms a second member (1002) of the device (100) and the controller (1016) is arranged to send the send the signal from the photodiode (1013) to the smart phone via the wireless communication device. The smart phone comprises a second controller, a second memory and a second wireless communication device. In use the second memory comprises code of an app. When running the app, the second controller is arranged to receive the signal from the first member (1001), to subtract the background readings, to extract the RR intervals and to run the algorithm to detect AF occurrences.

In a third embodiment (not shown), the device of the second exemplary embodiment further comprises a third member, a fourth member and a fifth member. The first member (1001), the third member, the fourth member and the fifth member are similar but each comprises a unique ID number stored in the memory. The first member (1001), the third member, the fourth member and the fifth member are each adhered to a different nail of the subject. In this example, the first member and the fourth member are adhered to nails of the right hand of the subject and the third member and the fifth member are adhered to nails of the left hand of the subject. The second controller is arranged to send instructions to the first member and the third member, such that the first member and the third member measure simultaneously.

The second controller is arranged to receive the signals from both the first member and the third member and to combine the signals to increase signal quality, for instance as the combination will be affected less by noise.

In case the controller of either first member or the third member detects an anomaly in its function, for instance diminished battery power, it sends an instruction to the second controller. Upon receiving the instruction, the second controller instructs the controller of the fourth or the fifth member to start measuring and instructs the member that detected the anomaly to stop measuring.

In a fourth exemplary embodiment (not shown), the member (1000) of the first exemplary embodiment does not comprise a pulse oximeter but a laser Doppler flowmetry (LDF) sensor. Laser Doppler flowmetry sensors are well known and comprise a radiation emitter and a detector for detecting reflected radiation. Laser Doppler flowmetry sensors measure a wavelength shift of laser radiation caused by moving blood cells to determine flow of microvascular blood. The flow of microvascular blood pulsates with the heart beats of a subject. Thus LDF sensors are optical sensors that measure a signal representing a blood perfusion parameter (flow) of the subject. In this example the first LED (1011), the second LED (1012) and the photodiode (1013) are replaced by a VCSEL to emit radiation and the detector and corresponding changes to the electrically conducting traces and through holes are made. In this example, no background measurements are taken.

In a fifth exemplary embodiment, the device (100) arranged to be adhered to the subjects big toe. This embodiment is illustrated in FIG. 3A, FIG. 3B and FIG. 3C.

The device (100) comprises a member (2000) that comprises a patch, in this case an adhesive band-aid in the form of a plaster that comprises a substrate (2001). On a bottom side, illustrated in FIG. 3C, the plaster comprises an area (2003) of adhesive material (2002). The area (2003) is in use adhered to the subjects toe nail. The substrate (2001) is made from a thin flexible material such as a flex PCB. The member comprises an optical sensor in the form of a pulse oximeter (OS). The optical sensor comprises a number of electronic components, here a first LED (1011), a second LED (1012), a photodiode (1013), a battery (1014), a memory (1015), a controller (1016) and a wireless communication device (1017). The electronic components are connected by electrically conducting traces (1018) to form a circuit. The battery (1014) is connected by the electrically conducting traces (1018) to all other electronic components to supply them with power and is arranged to supply power for a period of a week. In addition, the controller is connected to all other components, except the battery, to exchange commands and data such as the signal from the photodiode (1013) via additional electrically conducting traces (1019). The substrate (2001) comprises a first through hole (2031), a second through hole (2032) and a third through hole (2033). The first through hole (2031) is arranged to pass red light form the first LED (2031) to the nail (201). The second through hole (2032) is arranged to pass infrared radiation from the second LED (1032) to the nail (201). The third through hole is arranged to pass reflected red light and infrared radiation from the nail (201) and skin tissue (202) behind the nail (201) to the photodiode (1033).

The functionality of the pulse oximetry sensor is the same as in the first exemplary embodiment. The adherent material (2002) does not cover the complete side of the substrate (2001) that in use faces the subject. The area (2003) of the adherent material (2002) forms a ring positioned around a central area. The first through hole (1031), the second through hole (1032) and the third through hole (1033) end in the central area, i.e. they are not covered by and do not run through the adherent material (2002).

The first LED (1011), the second LED (1012) and the photodiode (1013) are positioned over the respective through holes (2031,2032,2033).

With the through holes (2031,2032,2033) ending in the central area surrounded by the area (2003) of adherent material (2002) and the first LED (1011,) the second LED (1012) and the third LED (1013) having such positions that they are located over the through holes ((2031,2032,2033), the adherent material (2002) and the optical sensor are arranged at such relative positions that the optical sensor operatively faces the nail in case the adherent material is fully adhered to the nail (201). In addition, the adherence of the plaster pulls the optical sensor toward the nail (201) which limits variation in distance between the optical sensor and the nail. Limiting the variation in distance between the optical sensor and the nail is advantageous to reduce noise on the signal from the optical sensor.

In a sixth exemplary embodiment of the invention a device (100) is arranged to be adhered to the subjects big toe. This embodiment is illustrated in FIG. 4A, FIG. 4B and FIG. 4C which are top views, side views and bottom views respectively.

The sixth embodiment is similar to the fifth embodiment. The difference is that a surface of the substrate (3001) that in use faces away from the subjects nail, is adhered to a first surface (3051) of a textile band-aid material (3050). The textile band-aid is ring shaped. An area (3003) of adhesive material (3002) is provided to adhere the device (100) to the subjects nail.

The adhesive material (3002) has a slightly smaller dimension in the direction perpendicular to the first surface (3051) than the substrate (3001).

The textile band-aid material (3050) is slightly elastic. However, the adherence of the adhesive material (3002) combined with the forces relating to elastic deformation of the band-aid material limit the varation of relative positions of the optical sensor and the subjects nail.

In an alternative embodiment, the textile band-aid material (3050) covers the complete device (100).

The invention may be implemented in embodiments differing from the exemplary embodiments described above. For instance in the exemplary embodiments described above, the optical sensors (laser Doppler flowmetry sensors or pulse oximetry sensors) were of a reflectance type (RPO), with both light emitter (LED) and detector on the same side of the finger or other body part(s).

In alternative embodiments, the sensors may be TPO type sensors. TPO type sensors are based on transmission through for example a finger; light emitter (LED) on one side and detector on the other side of the finger tip.

In alternative embodiments, the first LED or the second LED may be replaced by a Vertical Cavity Surface Emitting Laser (VCSEL).

The device may be a disposable or a non-disposable device and may be water resistible or not water resistible.

In alternative embodiments Suitable algorithms for RR extraction or AF detection can be selected in the understanding that the pulses measured as maxima in the ration of oxygenated and deoxygenated haemoglobin by the optical sensor relate to specific phases of the heart beats of the subject. In alternative embodiments, the device is booted by peeling off a layer. By peeling of a layer, a connection between the controller (1016) and the battery (1014) is severed and an input of the controller (1016) is no longer kept at a low voltage which triggers booting up the device.

### SUMMARY OF EMBODIMENTS

Below a summary of several embodiments (in use) is given. Where the embodiments refer to previous embodiments, this refers to previous embodiments in this summary.

### Embodiment 1

Atrial Fibrillation (AF) sensor integrated on or with a patch, characterized in that,

the sensor is an optical sensor and that the patch is arranged on a body part, e.g., finger, toe, ear, nose, wrist, forearm etc.

### Embodiment 2

AF sensor according to embodiment 1, characterized in that, that the sensor is laser doppler flowmetry sensor or a pulse oximetry sensor.

### Embodiment 3

AF sensor according to embodiment 1 or 2 characterized in that, that the sensor is either a reflective or a transmissive sensor.

### Embodiment 4

AF sensor according to any of the previous embodiments, characterized in that,

that the patch is arranged on a finger.

### Embodiment 5

AF sensor according any of the previous embodiments, characterized in that, that the patch is arranged on a body nail, e.g., finger or toe nail.

### Embodiment 6

AF sensor according any of the previous embodiments, characterized in that, that the patch is adhesive and arrangeable on a false nail attachable to the body nail.

### Embodiment 7

AF sensor according to any of embodiments 1 to 5, characterized in that, that the patch constitutes the false nail itself attachable to the body nail.

### Embodiment 8

AF sensor according to any of the previous embodiments, characterized in that,

that sensor components are miniaturized and integrated into the patch.

### Embodiment 9

AF sensor according to any of the previous embodiments, characterized in that,

that sensor is based on extraction of RR intervals from the optical signals and the detection of atrial fibrillation is based on the RR intervals.

### Embodiment 10

AF sensor according to any of the previous embodiments, characterized in that,

that the sensor functionality is split on several patches.

### Embodiment 11

AF sensor according to embodiment 10, characterized in that, that the patches are integrated on different or adjacent body nails.

### Embodiment 12

AF sensor according to embodiment 10, characterized in that, that the patches constitute false nails attachable to different body nails.

### Embodiment 13

AF sensor according to any of the previous embodiments, characterized in that,

that the patch is located on an arbitrary rigid place of the patient body, e.g. nail, false nail or bone area.

### Embodiment 14

AF sensor according to any of the previous embodiments, characterized in that,

that the sensor is located inside the patch.

The above embodiments should be regarded as illustrative rather than restrictive, and it should be appreciated that variations may be made in those embodiments by a person skilled in the art without departing from the scope of the present invention as defined in the following claims.

1. A device for detecting atrial fibrillation of a subject, comprising
a member including:
mechanical connection means to wearably connect the member to a subject; and
an optical sensor arranged to measure a signal representing a blood perfusion parameter of the subject;
the optical sensor and mechanical connection means located at such relative positions that when the mechanical connection means connects the member to the subject, the optical sensor operatively faces the subject;
wherein the device is configured to detect atrial fibrillation of the subject based on a signal measured by the optical sensor.

2. The device according to claim 1, wherein the optical sensor is a laser Doppler flowmetry sensor or a pulse oximetry sensor.

3. The device according to claim 1, wherein the mechanical connection means includes an area of adherent material arranged to adhere the member to the subject.

4. The device according to claim 3, wherein the adherent material is configured to adhere to a nail or false nail of the subject; the optical sensor and the adherent material being arranged at such relative positions that when the adherent material adheres to a nail or false nail of the subject, the optical sensor operatively faces the nail or false nail and is pulled towards the nail or false nail by the adherent material.

5. The device according to Device of claim 4, wherein the optical sensor and the adherent material are arranged in such relative positions that in use the relative positions of the nail or false nail and the optical sensor are fixed.

6. The device according to claim 5, wherein the device further comprises a false nail.

7. The device according to claim 1, further comprising a processing unit configured to extract RR intervals from the signal and to detect atrial fibrillation based on the extracted RR intervals.

8. The device according to claim 1, wherein the device includes a plurality of members, a first member of the plurality of members including the optical sensor; wherein the functionality of the device is split over the plurality of members.

9. The device according to claim 8, wherein each member of the plurality of members includes an individual area of adherent material arranged to adhere the corresponding member to the subject.

10. The device according to claim 9, wherein each of the plurality of members includes comprises a false nail.

11. The device according to claim 8, wherein the plurality of members includes a second member, the second member including a processing unit arranged to process the signal for detecting atrial fibrillation.
